SEQUENCE LISTING (3 sequences)

SEQ ID NO: 1 - RNA, 21 nt, Brassica oleracea
agaaucuugaugaugcugcau

SEQ ID NO: 2 - RNA, 21 nt, Brassica oleracea
agaaucuugaugaugcugcau

SEQ ID NO: 3 - RNA, 3582 nt, Homo sapiens
gccggaguccccacgcgaggaugcugcggugagcgcggccgggacgccgcgucgggcucucggccgcccagcggcgccgcaggggaagccaggccggcagccgcgcgcuccccagccggccaccaaucccgcccuccccggcuccuccggaccuccucggcaggcggcggcggggccugccugugcgcucugcgcccgcccgcgccuacccuccauggcguuuauccggaagaagcagcaggagcagcagcugcagcucuacuccaaggagagauuuuccuugcugcugcuuaacuuggaggaguacuacuuugaacagcauagagccaaucacauuuugcacaagggcagucaccaugaaaggaaaaucagaggcuccuuaaaaauauguucaaaaucggugauuuuugaaccagauucaauaucccagcccaucaucaagauuccuuugagagacuguauaaaaauaggaaagcauggagaaaauggagccaauagacacuucacaaaggcaaaaucuggggguauuucacucauuuucagucagguauauuucauuaaagaacauaauguuguugcaccauauaaaauagaaaggggcaaaauggaauauguuuuugaauuggauguucccgggaaaguggaagauguuguggagacguugcuucagcuucacagagcauccugccuugacaaauugggugaccaaaccgccaugauaacagcuauuuugcagucucguuuagcuagaacaucauuugacaaaaacagguuccaaaacauuucugaaaagcugcacauggaaugcaaagcagaaauggugacgccucuggugacuaauccuggacacgugugcaucacggacacaaaccuguauuuucagccccucaacggcuacccgaaaccugugguccagauaacacuccaagauguccgccgcaucuacaaaaggaggcacggccucaugccucugggcuuggaaguauuuugcacagaagaugaucuguguuccgacaucuaccuaaaguucuaugaaccucaagauagagaugaucucuauuuuuacauugccacauaccuagagcaccauguggcggagcacacugcugagagcuacaugcugcaguggcagcguggacaccuuuccaacuaucaguaccuccuucaccucaacaaccuggccgaccgcagcugcaacgaccucucccaguacccuguguuuccauggauaauacaugauuauuccagcucagaacuagauuugucaaauccaggaaccuuccgggaucucaguaagccaguaggggcccuaaauaaggaacggcuggagagacuacugacacgcuaccaggaaaugccugaaccaaaguucauguaugggagucacuacucuuccccggguuauguacuuuuuuaucuuguuaggauugcaccagaguauaugcugugccugcagaauggaagauuugauaaugcagauagaauguucaacaguauugcagaaacuuggaaaaacugucuggauggugcaacggauuuuaaagaguuaauuccagaauucuauggugaugaugugagcuuucuagucaauagccugaaguuggauuugggaaagagacaaggaggacagaugguugacgacguggagcuucccccuugggcuuccagucccgaggacuuucuccagaagagcaaagaugcauuggaaagcaauuaugugucugaacaccuucacgaguggauugaucuaauauuuggcuacaaacaaaaagggagugaugcaguuggggcccauaauguauuucauccccugaccuaugaaggagguguagacuugaacagcauccaggauccugaugagaagguagccaugcuuacgcaaaucuuggaauuugggcagacaccaaaacaacuauuugugacaccacauccucgaaggaucaccccaaaguuuaaaaguuugucccagaccuccaguuauaaugcuucuauggcagauuccccaggugaagagucuuuugaagaccugaccgaagaaagcaaaacacuggccuggaauaacaucaccaaacugcaguuacacgagcacuauaaaauccacaaagaagcaguuacuggaaucacggucucucgcaauggaucuucaguauucacaacaucccaagauuccaccuugaagauguuuucuaaagaaucaaaaaugcuacaaagaaguauaucauuuucaaauauggcuuuaucgucuuguuuacuuuuaccaggagaugccacugucauaacuucuucaugggauaauaaugucuauuuuuauuccauagcauuuggaagacgccaggacacguuaaugggacaugaugaugcuguuaguaagaucuguuggcaugacaacaggcuauauucugcaucgugggacucuacagugaagguguggucugguguuccugcagagaugccaggcaccaaaagacaccacuuugacuugcuggccgagcuggaacaugaugucaguguagauacaaucaguuuaaaugcugcaagcacacuguuaguuuccggcaccaaagaaggcacagugaauauuugggaccucacaacggccaccuuaaugcaccagauuccaugccauucagggauuguaugugacacugcuuuuagcccagauagucgccauguccucagcacaggaacagauggcugucuuaaugucauugaugugcagacaggaaugcucaucuccuccaugacaucagaugagccccagaggugcuuugucugggauggaaauuccguuuuaucuggcagucagucuggugaacugcucguuugggaccuccuuggagcaaaaaucagugagagaauacagggccacacaggugcugugacauguauauggaugaaugaacaguguagcaguaucaucacaggaggggaagacagacaaauuauauucuggaaauugcaguauuaagugccuuuuccucuccugaauauuaaauugaacucuauuuaaugcauuuuuaaaccaaacuuuuaaacggacuggugaaugugcaauguuaguaauuagaaguuuuaccacauggaaaauuugugguuuuaaacuuucuaaaucauggugacuucauugaaagccauuaguugcuauucucuuagggcagauaaaaugcggcuguguuaggaaaaacauguuacacuguaaggcagaugaucguccccguaugaugauugucagaagacaggacuaaguagcagagaauagcuaagagauaaauugggcuggggaaacuugucagaaagcacugaacaauuaagaaauuuuccaagaaaaugugcaguauucucugcuacuucugaaucuguuuugucuuccuaaucuaucacaauugccacccaucggguuuuggguguguguuuucauagcgugguuacuuucuauaaugcuguacccagauucuaagaaccuggagaaggauuagcaguucuuaguaaguuuacuguguauaggaacgguuuguauuucauuacagcuauucaucuuuucuacauuaaaaauauuuuucucuaaagaaaaaaaaaaaaaaa

(19) United States
(12) Patent Application Publication
(10) Pub. No.: US 2016/0272966 A1
(43) Pub. Date: Sep. 22, 2016

(54) Method of using a miR172 Molecule for Decreasing Inflammation

(71) Applicants: INSTYTUT BIOCHEMII I BIOFIZYKI PAN, Warzawa (PL); UNIWERSYTET IM. ADAMA MICKIEWICZA W POZNANIU, Poznan (PL)

(72) Inventors: Anna Lukasik, Konin (PL); Piotr Zielenkiewicz, Warszawa (PL); Zofia Szweykowska-Kulinska, Poznan (PL); Leszek Paczek, Warszawa (PL); Maria Nowaczyk, Warszawa (PL)

(21) Appl. No.: 14/913,650

(22) PCT Filed: Aug. 13, 2014
(86) PCT No.: PCT/PL2014/000092

§ 371 Date: Feb. 22, 2016

(30) Foreign Application Priority Data
Aug. 23, 2013 (PL) PL405125

(51) Int. Cl. C12N 15/113

(52) U.S. Cl. CPC C12N 15/113; C12N 2310/141

(57) ABSTRACT

An exemplary embodiment relates to a method of using a plant-derived miR172 molecule or its synthetic equivalent, selected from amongst miR172a or miR172b, for decreasing inflammatory processes in an organism, a method of decreasing B and T lymphocyte proliferation, as well as a method of reducing protein FAN (Factor Associated with Neutral Sphingomyelinase Activation) level. Exemplary embodiments provide a novel therapeutic method based on miRNA molecules, which through the interaction with mRNA encoding FAN protein, negatively regulate its expression and decrease the inflammatory response of the organism.

### TECHNICAL FIELD

An exemplary embodiment relates to a method of using a plant-derived miR172 molecule or its synthetic equivalent, selected from amongst miR172a or miR172b, for decreasing inflammatory processes in an organism, a method of decreasing B and T lymphocyte proliferation, as well as a method of reducing protein FAN (Factor Associated with Neutral Sphingomyelinase Activation) level. Exemplary embodiments deliver a novel therapeutic method based on miRNA molecules, which through the interaction with mRNA encoding FAN protein, negatively regulate its expression and decrease the inflammatory response of the organism.

### BACKGROUND

The state of the relevant art as is known to Applicant is described in the following. The disclosure of each of which is incorporated by reference herein in its entirety.

In the patent application CN102887948 antibacterial and immunoregulation polypeptide medicine is described. The disclosure belongs to a biotechnology, and relates to an antibacterial and immunoregulation apoE-mimetic peptide medicine. The apoE-mimetic peptide ApoE23 has a structure represented by the sequence1. In vivo and in vitro tests demonstrate that the apoE-mimetic peptide can lower the LPS-induced TNF-alpha, IL-6 and IL-10 expression in THP-1 cells and human peripheral blood mononuclear cells, obviously reduce the death rate of mice with septicopyemia, reduce the TNF-α, IL-6 and LPS concentrations in the blood plasma of the mice with septicopyemia, relieve the inflammation in the lung, liver, small intestine and spleen of the mice with septicopyemia, and can kill Escherichia coli, Pseudomonas aeruginosa, pan-drug resistant Acinetobacter baumannii and Staphylococcus aureus. The apoE-mimetic peptide can be used to prepare Gram-negative bacilli resistant and pan-drug resistant Gram-negative bacilli resistant polypeptide medicines and immunoregulation polypeptide medicines and anti-septicopyemia medicines.

Patent application WO2012153854 provides means for modulating the expression of cytokine-chemokine genes associated with inflammation and infection are described. Specifically, the disclosure provides an antisense nucleotide that comprises the sequence complementary to the 3′UTR of the CCL2, CCL20, CX3CL1, IL-23A, CD69, NF-κB p65, TNF-α, Fam89a, Grk5, phosopholipid scramblase 1, Runx1, semaphorin 4A, Steap4, lymphotoxin β Psmb10 or TLR2 gene induced by IL-1β and that is capable of modulating the expression of the gene; sense oligonucleotide that contains the sequence complementary to the endogenous antisense transcription product containing the sequence complementary to the 3′UTR of the gene and that is capable of modulating the expression of the gene; and an agent that prevents and/or treats inflammatory disease or infection and contains the sense or antisense nucleotide.

In the patent application AU2012201409 RNA interference is provided for inhibition of tumor necrosis factor α (TNF-α) by silencing TNF-α cell surface receptor TNF receptor-I (TNFRI) mRNA expression, or by silencing 5 TNF-α converting enzyme (TACE/ADAM17) mRNA expression. Silencing such TNF-α targets, in particular, is useful for treating patients having a TNF-α-related condition or a risk of developing a TNF-α-related condition such as the ocular conditions dry eye, allergic conjunctivitis, or ocular inflammation, or such as dermatitis, rhinitis, or asthma, for example.

Patent application WO2012115469 relates to an anti-inflammatory functional food composition for oral administration, and more particularly, to a method for extracting, from grain and fruit, active ingredients which are effective in preventing inflammation and improving symptoms, and to an anti-inflammatory functional food composition comprising the extracts for oral administration. To this end, the anti-inflammatory functional food composition for oral administration according to the disclosure is prepared by obtaining extracts of Morus alba L. (mulberry), black turtle beans (black beans), and onions, and mixing said extracts at an appropriate ratio. The thus-prepared functional food composition may be used as a composition for anti-inflammatory analgesics, and has the effects of preventing and alleviating the symptoms of arthritis, as it exhibits, in an appropriate capacity range, superior antioxidant effects, inflammatory edema relieving effects, acute inflammation and chronic inflammation inhibitory effects, effects of inhibiting the expression of lipoxygenase, cyclooxygenase-2, inducible nitric oxide synthase (iNOS) and type I collagenase which are pro-inflammatory enzymes, and effects of inhibiting the production of tumor necrosis factor-alpha (TNF-α), interleukin 1-beta (IL-1β), interleukin 6 (IL-6) and prostaglandins, which are pro-inflammatory factors.

In a patent application KR101090177 an IL-32 antagonistic RNA aptamer is provided to suppress TNF-α expression and to develop a therapeutic agent for inflammatory diseases. An RNA aptamer has a sequence of sequence number 7 and binds to IL-32 as an antagonist. The RNA aptamer blocks the function of intrinsic IL-32. The RNA aptamer has high affinity with IL-32. The RNA aptamer is applied for developing a therapeutic agent for treating chronic inflammation such as rheumatic arthritis.

The patent application JP2003267880 describes composition for antiallergy and antiinflammation, which has excellent stability, cost, and is safe. The interleukin-4 production inhibitor comprises Brassica oleracea acephala and/or Brassica orelacea acephala extract. The composition for antiallergy and the composition for antiinflammation comprise the interleukin-4 production inhibitor and have excellent effect on allergies such as allergic rhinitis, pollinosis, etc. These compositions are orally administered or used as skin care preparations.

Patent application WO03070897 concerns methods and reagents useful in modulating TNF superfamily and TNF receptor superfamily gene expression in a variety of applications, including use in therapeutic, diagnostic, target validation, and genomic discovery applications. Specifically, the disclosure relates to small nucleic acid molecules, such as short interfering nucleic acid (siNA), short interfering RNA (siRNA), double-stranded RNA (dsRNA), micro-RNA (miRNA), and short hairpin RNA (shRNA) molecules capable of mediating RNA interference (RNAi) against TNF superfamily and TNF receptor superfamily gene expression and/or activity. The small nucleic acid molecules are useful in the treatment of septic shock, rheumatoid arthritis, HIV and AIDS, psoriasis, inflammatory or autoimmune disorders and any other disease or condition that responds to modulation of TNF and/or TNF receptor expression or activity.

In the patent application US2008279862 methods for treating an inflammatory or an immune condition are described. The disclosure relates to methods for treating an inflammatory or an immune condition with IL-1 inhibitors and an inhibitor of B cell or T cell activation. Methods for treating an inflammatory or an immune condition with TNF inhibitors and an inhibitor of B cell or T cell activation are described.

In the patent application US2012301484 regulatory T cell proteins and uses thereof are described. One protein, designated PD-L3, resembles members of the PD-L1 family, and co-stimulates αCD3 proliferation of T cells in vitro. A second, TNF-like, protein has also been identified as being upregulated upon αCD3/αGITR stimulation. This protein has been designated T^{reg}-sTNF. Proteins, antibodies, activated T cells and methods for using the same are disclosed. In particular methods of using these proteins and compounds, preferably antibodies, which bind or modulate (agonize or antagonize) the activity of these proteins, as immune modulators and for the treatment of cancer, autoimmune disease, allergy, infection and inflammatory conditions, e.g. multiple sclerosis is disclosed.

Patent application DE4006768 relates to the antirheumatic agent (I) obtained from the juice of cabbage leaves, which also contains olive oil to give the mixture the right consistency. Preferably it is in the form of a salve, which contains (in addition to the olive oil) an emulsifier and a preservative. A preferred ratio: 10-80 wt. % leaf extract/juice and 10-80 wt. % olive oil; ideally the salve contains a total of 5-80 (10-40) wt. % olive oil and emulsifier. Preferably the olive oil is cold-pressed. The antirheumatic agent (I) alleviates inflammation and pain associated with rheumatism. It can also be used in cases of lumbago, migraine, headache, tendon inflammation, dislocation and swelling. If necessary the salve can be wrapped in tissues and used overnight as a compress.

In the patent application PL397846 method of treating TNF-α-related diseases, comprising administering of TNF-α inhibitors, including TNF-α antibodies is described

The following publications are also each incorporated by reference in there entirety:
1. Liu N, Okamura K, Tyler D M, Phillips M D, Chung W J, Lai E C: The evolution and functional diversification of animal microRNA genes. Cell research 2008, 18(10):985-996.
2. Jones-Rhoades M W: Conservation and divergence in plant microRNAs. Plant molecular biology 2012, 80(1):3-16.
3. Niwa R, Slack F J: The evolution of animal microRNA function. Current opinion in genetics & development 2007, 17(2):145-150.
4. Djuranovic S, Nahvi A, Green R. miRNA-mediated gene silencing by translational repression followed by mRNA deadenylation and decay. Science 2012, 336(6078):237-240.
5. Hu W, Coller J: What comes first: translational repression or mRNA degradation? The deepening mystery of microRNA function. Cell research 2012, 22(9):1322-1324.
6. Ying S Y, Chang D C, Lin S L: The microRNA (miRNA): overview of the RNA genes that modulate gene function. Molecular biotechnology 2008, 38(3):257-268.
7. Yang Z, Ebright Y W, Yu B, Chen X: HEN1 recognizes 21-24 nt small RNA duplexes and deposits a methyl group onto the 2′ OH of the 3′ terminal nucleotide. Nucleic acids research 2006, 34(2):667-675.
8. Bartel D P: MicroRNAs: genomics, biogenesis, mechanism, and function. Cell 2004, 116(2):281-297.
9. Chen X: MicroRNA biogenesis and function in plants. FEBS letters 2005, 579(26):5923-5931.
10. Kim V N, Han J, Siomi M C: Biogenesis of small RNAs in animals. Nature reviews Molecular cell biology 2009, 10(2):126-139.
11. Khraiwesh B, Arif M A, Seumel G I, Ossowski S, Weigel D, Reski R, Frank W: Transcriptional control of gene expression by microRNAs. Cell 2010, 140(1):111-122.
12. Kawamata T, Tomari Y: Making RISC. Trends in biochemical sciences 2010, 35(7):368-376.
13. Sontheimer E J: Assembly and function of RNA silencing complexes. Nature reviews Molecular cell biology 2005, 6(2):127-138.
14. Zhang B, Wang Q, Pan X: MicroRNAs and their regulatory roles in animals and plants. Journal of cellular physiology 2007, 210(2):279-289.
15. Wienholds E, Plasterk R H: MicroRNA function in animal development. FEBS letters 2005, 579(26):5911-5922.
16. Esteller M: Non-coding RNAs in human disease. Nature reviews Genetics 2011, 12(12):861-874.
17. De Guire V, Robitaille R, Tetreault N, Guerin R, Menard C, Bambace N, Sapieha P: Circulating miRNAs as sensitive and specific biomarkers for the diagnosis and monitoring of human diseases: promises and challenges. Clinical biochemistry 2013, 46(10-11):846-860.
18. Broderick J A, Zamore P D: MicroRNA therapeutics. Gene therapy 2011, 18(12):1104-1110.
19. Mack G S: MicroRNA gets down to business. Nature biotechnology 2007, 25(6):631-638.
20. Xiao C, Rajewsky K: MicroRNA control in the immune system: basic principles. Cell 2009, 136(1):26-36.
21. Hukowska-Szematowicz B, Tokarz-Deptula B, Deptula W: MicroRNA (miRNA) and the immune system. Centr Eur J Immunol 2012, 37(4):387-390.
22. Liu G, Abraham E: MicroRNAs in immune response and macrophage polarization. Arteriosclerosis, thrombosis, and vascular biology 2013, 33(2):170-177.
23. Filkova M, Jungel A, Gay R E, Gay S: MicroRNAs in rheumatoid arthritis: potential role in diagnosis and therapy. BioDrugs: clinical immunotherapeutics, biopharmaceuticals and gene therapy 2012, 26(3):131-141.
24. Boissier M C, Semerano L, Challal S, Saidenberg-Kermanac'h N, Falgarone G: Rheumatoid arthritis: from autoimmunity to synovitis and joint destruction. Journal of autoimmunity 2012, 39(3):222-228.
25. Simoni Y, Diana J, Ghazarian L, Beaudoin L, Lehuen A: Therapeutic manipulation of natural killer (NK) T cells in autoimmunity: are we close to reality? Clinical and experimental immunology 2013, 171(1):8-19.
26. Fournier C: Where do T cells stand in rheumatoid arthritis? Joint, bone, spine: revue du rhumatisme 2005, 72(6):527-532.
27. Rickert R C, Jellusova J, Miletic A V: Signaling by the tumor necrosis factor receptor superfamily in B-cell biology and disease. Immunological reviews 2011, 244(1):115-133
28. Stinissen P, Raus J, Zhang J: Autoimmune pathogenesis of multiple sclerosis: role of autoreactive T lymphocytes and new immunotherapeutic strategies. Critical reviews in immunology 1997, 17(1):33-75.
29. Krueger J G, Bowcock A: Psoriasis pathophysiology: current concepts of pathogenesis. Annals of the rheumatic diseases 2005, 64 Suppl 2:ii30-36.
30. Campbell K A, Lipinski M J, Doran A C, Skaflen M D, Fuster V, McNamara C A: Lymphocytes and the adventitial immune response in atherosclerosis. Circulation research 2012, 110(6):889-900.
31. Youinou P, Jamin C, Saraux A: B-cell: a logical target for treatment of rheumatoid arthritis. Clinical and experimental rheumatology 2007, 25(2):318-328.
32. Huseyin T E Ozer, Ozbalkan Z: Clinical Efficacy of TNF-α Inhibitors: An Update. International Journal of Clinical Rheumatology 2010, 5(1):101-115.
33. Croft M, Benedict C A, Ware C F: Clinical targeting of the TNF and TNFR superfamilies. Nature reviews Drug discovery 2013, 12(2):147-168.
34. Montfort A, Martin P G, Levade T, Benoist H, Segui B: FAN (factor associated with neutral sphingomyelinase activation), a moonlighting protein in TNF-R1 signaling. Journal of leukocyte biology 2010, 88(5):897-903.
35. Palin K, Bluthe R M, McCusker R H, Levade T, Moos F, Dantzer R, Kelley K W: The type 1 TNF receptor and its associated adapter protein, FAN, are required for TNFalpha-induced sickness behavior. Psychopharmacology 2009, 201(4):549-556.
36. Montfort A, de Badts B, Douin-Echinard V, Martin P G, Iacovoni J, Nevoit C, Therville N, Garcia V, Bertrand M A, Bessieres M H et al: FAN stimulates TNF(alpha)-induced gene expression, leukocyte recruitment, and humoral response. J Immunol 2009, 183(8):5369-5378.
37. Challem J: The Inflammation Syndrome: The Complete Nutritional Program to Prevent and Reverse Heart Disease, Arthritis, Diabetes, Allergies, and Asthma: John Wiley & Sons; 2003.
38. Pizzorno J E J, Murray M T, Joiner-Bey H: The Clinician's Handbook of Natural Medicine: Elsevier Health Sciences; 2008.
39. Torkos S: The Canadian Encyclopedia of Natural Medicine: John Wiley & Son; 2012.
40. Carper J: Apteka zywnosci. Nowe i niezwykle odkrycia leczniczego dzialania zywnosci: Vesper; 2008.
41. Gornicka J: Apteka natury: poradnik zdrowia: ziololecznictwo, akupresura, masaz shiatsu: AWM; 1997.
42. Munns A: Cabbage leaves: cabbage leaves can help inflammation of any body part. BMJ 2003, 327(7412):451.
43. Zhang L, Hou D, Chen X, Li D, Zhu L, Zhang Y, Li J, Bian Z, Liang X, Cai X et al: Exogenous plant MIR168a specifically targets mammalian LDLRAP1: evidence of cross-kingdom regulation by microRNA. Cell Res 2012, 22(1):107-126.
44. Wang K, Li H, Yuan Y, Etheridge A, Zhou Y, Huang D, Wilmes P, Galas D: The complex exogenous RNA spectra in human plasma: an interface with human gut biota? PLoS One 2012, 7(12):e51009.
45. Szarzynska B, Sobkowiak L, Pant B D, Balazadeh S, Scheible W R, Mueller-Roeber B, Jarmolowski A, Szweykowska-Kulinska Z: Gene structures and processing of Arabidopsis thaliana HYL1-dependent pri-miRNAs. Nucleic acids research 2009, 37(9):3083-3093.
46. Lukasik A, Pietrykowska H, Paczek L, Szweykowska-Kulinska Z, Zielenkiewicz P: High-throughput sequencing identification of novel and conserved miRNAs in the Brassica oleracea leaves. BMC genomics 2013, 14:801.
47. Pruitt K D, Harrow J, Harte R A, Wallin C, Diekhans M, Maglott D R, Searle S, Farrell C M, Loveland J E, Ruef B J et al: The consensus coding sequence (CCDS) project: Identifying a common protein-coding gene set for the human and mouse genomes. Genome research 2009, 19(7):1316-1323.
48. Enright A J, John B, Gaul U, Tuschl T, Sander C, Marks D S: MicroRNA targets in Drosophila. Genome biology 2003, 5(1):R1.
49. Conesa A, Gotz S, Garcia-Gomez J M, Terol J, Talon M, Robles M: Blast2GO: a universal tool for annotation, visualization and analysis in functional genomics research. Bioinformatics 2005, 21(18):3674-3676.

### SUMMARY

Exemplary embodiments provide methods of treating various types of disorders and conditions by decreasing the inflammatory response of an organism by using plant based miR172a or miR172b molecules, or their synthetic equivalent. In an exemplary embodiment, the plant miR172a and miR172b molecules are represented, respectively, by bol-miR172a and bol-miR172b molecules derived from Brassica oleracea var. capitata (cabbage). In exemplary embodiments, auto immune diseases may be treated with miR172a or miR172b molecules.

### BRIEF DESCRIPTION OF THE DRAWINGS

FIG. 1 shows the binding site of mRNA encoding FAN protein (marked as “Reference”) predicted for bol-miR172a molecule (marked as “Query”).

FIG. 2 shows the results of the measurements of FAN protein concentration (ng/ml) depending on the bol-miR172a molecule presence (at 3 different concentrations).

FIG. 3 shows the results of the in vitro assay for the T and B lymphocyte function.

FIG. 3a shows the influence of the bol-miR172a molecule (present at 3 different concentrations) on the proliferation of T lymphocytes stimulated by phytohemagglutinin (PHA).

FIG. 3b shows the influence of the bol-miR172a (present at 3 different concentrations) on the proliferation of B lymphocytes stimulated by Staphylococcus aureus (Cowan strain, SAC).

FIG. 3c shows the influence of the bol-miR172a (present at 3 different concentrations) on the proliferation of T lymphocytes stimulated by anti-CD3 antibodies (OKT3).

FIG. 3d shows the influence of the bol-miR172a (present at 3 different concentrations) on the proliferation of B lymphocytes in the case of their autostimulation.

FIG. 4 shows the results of the phenotypic evaluation of PBMCs using flow cytometry.

FIG. 4a shows the results of phenotypic evaluation of MNCs incubated with miRNA and miRNA+PHA mitogen for 3 hours.

FIG. 4b shows the results of phenotypic evaluation of MNCs incubated with miRNA and miRNA+PHA mitogen for 6 hours.

FIG. 4c shows the results of phenotypic evaluation of MNCs incubated with miRNA and miRNA+PHA mitogen for 24 hours.

### DETAILED DESCRIPTION

MicroRNA (miRNA) are a class of single-stranded, regulatory RNAs that are widely evolutionarily conserved among many known species. These short (18-24 nt), non-coding molecules mediate post-transcriptional gene regulation by promoting cleavage or inhibiting translation of the target mRNA. As a mature sequence forms, miRNAs are generated in multi-step process, which begins with miRNA gene transcription into long primary transcript with many stem-loop units (pri-miRNA). The pri-miRNA is further processed into the hairpin precursor (pre-miRNA) and cleaved to generate miRNA:miRNA* duplex with two nucleotide overhangs at the 3′ end.

Individual stages of miRNAs precursors processing are slightly different in animal and plant organisms. However, the final step of miRNAs maturation process is similar in both cases. One of the duplex strands (*-strand) is usually degraded; whereas the second strand is loaded on the RISC (RNA-Induced Silencing Complex) multi-complex, which binds to the specific mRNA transcript. Through this hybridization, miRNAs negatively regulate the expression of target genes thereby controlling cell development, apoptosis, proliferation, differentiation and other important functions in living organisms.

In humans, several reports have associated the expression profiles of specific miRNAs with certain physiological and pathological stages, tumorigenesis or even patient response to treatment. Thus, miRNAs serve as diagnostic and prognostic biomarkers in medicine, and have been incorporated in therapies for treating several human disorders. The miRNAs are also involved in the regulation of organism immune response, immunity or inflammation. miRNAs' effect on these processes is connected among others with the: (1) interferon and cytokines signalling pathways, (2) development and differentiation regulation of B and T lymphocytes, macrophages, monocytes, NK cells, as well as granulocytes, or (3) activation of some of these cells, which are part of innate and adaptive immune system. The aforementioned T and B lymphocytes and such factors as the tumor necrosis factor alpha (TNF-α) play an important and critical, role in a number of autoimmune diseases. These diseases may include, but are not limited to rheumatoid arthritis, psoriasis, atherosclerosis, Crohn's disease and multiple sclerosis. There are many known methods to treat autoimmune diseases. However, there are limitations due to their significant adverse effects, which include increased susceptibility to infections.

A therapeutic target in the treatment of autoimmune diseases is the Factor Associated with Neutral Sphingomyelinase Activation (FAN) protein encoded by the NSMAF gene. FAN is an adaptor molecule, which, through its interaction with the NSD domain of the TNF-R1 receptor, modulates expression of genes induced by the TNF-α; namely, genes encoding inflammatory proteins, such as interleukin 6 (IL-6), and chemokines, e.g., CCL5, CCL9, CCL20 and CXCL2. Research performed on mice showed that in FAN^{-/-} individuals, the TNF-α-induced expression of the aforementioned pro-inflammatory molecules was selectively altered. This resulted in a disruption of the leukocyte population in secondary lymphoid organs, as observed through the reduction of macrophages, neutrophils, dendritic and lymphoid cells number in the spleen.

Aside from all drug treatments, there are also household methods of dealing with inflammation and immune responses. One of these methods is the use of cabbage (Brassica oleracea var. capitata), and more precisely applying cabbage leaves compresses or drinking the cabbage juice. Due to its specific properties, cabbage may be used in natural medicine mainly for rheumatic pain, veins and lymphatic vessels inflammation, bruises, sprains, mastitis, or gastrointestinal problems. Its “spectrum” of uses may and encompass treatment of both internal and external diseases. The effect of consumed plants on the human organism may be attributed not only to the minerals, antioxidants, vitamins or polypeptides contained therein, but also to the presence of small RNA molecules—miRNAs. The study by Zhang et al. provided evidence not only that food-derived miRNAs are abundant in human serum but also that they can negatively regulate expression of specific genes in mammals. For example, MIR168a inhibits expression of the low-density lipoprotein receptor adapter protein 1 (LDLRAP1) in liver and thereby disrupts LDL plasma homeostasis. Plant-derived miRNAs were also identified by other studies which showed that miRNA molecules compose a significant sRNAs fraction in human plasma. The cross-kingdom regulation by plant miRNAs provides additional opportunities fir natural medicine in the treatment of many known diseases.

An exemplary embodiment comprises a method of treating various types of disorders and conditions by decreasing the inflammatory response of an organism by using plant based miR172a or miR172b molecules, or their synthetic equivalent. In an exemplary embodiment, the plant miR172a and miR172b molecules are represented, respectively, by bol-miR172a and bol-miR172b molecules derived from Brassica oleracea var. capitata (cabbage). Bioinformatics analysis of the results obtained by high-throughput sequencing of Brassica oleracea sRNAs was carried out, which confirmed the presence and determined the relative quantity of miRNA molecules in mature cabbage leaves. In an exemplary embodiment, the prediction of human target genes for identified cabbage miRNAs was performed.

The aforementioned bioinformatics target genes prediction for B. oleracea miRNAs revealed that bol-miR172a and bol-miR172b molecules, present in considerable quantities in cabbage leaves, may interact with mRNA encoding FAN protein and therefore negatively regulate its expression. The FAN protein mediates inflammatory responses by interacting with tumor necrosis factor receptor-1 (TNF-R1) and for this reason become a potential therapeutic target in the treatment of autoimmune diseases. Measurements of FAN protein quantity performed on PBMCs (Peripheral Blood Mononuclear Cells) with the use of the ELISA assay confirmed that the presence of bol-miR172a or bol-miR172b reduces the level of FAN protein. Moreover, an in vitro test assessing the function of T and B lymphocytes under the stimulation of specific mitogens revealed that abundance of the aforementioned miRNAs molecules (at an appropriate concentration) decrease T and B lymphocytes proliferation. Phenotypic evaluation of PBMCs showed that the presence of bol-miR172a, as well as simultaneous presence of bol-miR172a and specific mitogen, alters fraction of CD25+ cells in lymphocyte CD3+ population.

Exemplary embodiments provide for the use of miRNA molecules for the regulation of inflammatory processes in the human organism and provide methods of utilising said molecules for therapeutic purposes.

An exemplary method includes a use of a plant-derived miR172 molecule or its synthetic equivalent, selected from amongst miR172a or miR172b, wherein miR172a is represented by the sequence SEQ. ID No. 1 and miR172b by the sequence SEQ. ID No. 2, and wherein said molecule has at least 6 out of 7 nucleotides present in the “seed” region represented by the sequence GAAUCUU and has at least 75% sequence similarity to SEQ. ID No. 1 or SEQ. ID No. 2, for decreasing the inflammatory response or for preventing an increase in the inflammatory response of an organism.

An exemplary method includes a use for reducing proliferation of T and B lymphocytes.

An exemplary method includes a use for reducing the level of FAN (Factor Associated with Neutral Sphingomyelinase Activation) protein.

An exemplary method includes a use wherein plant-derived miR172 molecule or its synthetic equivalent interacts with mRNA encoding FAN protein negatively regulating its expression and therefore reducing proliferation of T and B lymphocytes.

An exemplary method includes a use wherein plant-derived miR172 molecule or its synthetic equivalent interacts with mRNA encoding FAN protein negatively regulating its expression and therefore reducing the level of FAN protein.

An exemplary method includes a use wherein expression of the FAN protein is negatively regulated and the level of FAN protein is reduced.

An exemplary method includes a use wherein the molecule is 6, 7, 8, 9, 10, 11, 12, 13, 14, 15, 16, 17, 18, 19, 20, 21, 22, 23, 24, 25, 26, 27 or 28 nucleotides long.

An exemplary method includes a use wherein the molecule has 2′- or 3′-O-methylation of the ribose of last nucleotide at the 3′ end.

An exemplary method includes a use wherein the inflammatory response of the organism is decreased by reducing proliferation of T and B lymphocytes.

An exemplary method includes a use wherein the inflammatory response of the organism is decreased by reducing the level of FAN protein.

In vitro lymphocyte proliferation assay were performed, which measures lymphocytes ability to proliferate in response to various stimuli, showed that presence of bol-miR172a molecule reduces the proliferation of B lymphocytes, as well as T lymphocytes in the case of their autostimulation or stimulation by SAC, OKT3 and PHA mitogen, respectively. Moreover, the phenotype evaluation of PBMCs using flow cytometry method revealed that abundance of the bol-miR172a molecule, with or without the simultaneous presence of the PHA mitogen, slightly changes the fraction of the CD25+ cells in T cell population. Results showed that bol-miR172a and bol-miR172b molecules are able to modulate B and T lymphocyte proliferation without affecting the activated T cells (CD25+) subpopulation. The mechanism responsible for the presented actions of bol-miR172a and bol-miR172b molecules is related to their interaction with mRNA encoding FAN protein. The influence of bol-miR172a molecule on the FAN protein level in PBMCs was evaluated by in vitro ELISA assay. Performed analysis revealed that abundance of the bol-miR172a (in all tested concentrations) results in a significant reduction of the FAN protein level.

Administering the molecule may include various routes, but is not limited to the oral administration route such as, swallowed by mouth as a pill, liquid, tablet or lozenge, the rectal route such as a suppository inserted into the rectum, an intravenous rout such as being injected into vein with a syringe or into intravenous (IV) line, an infusion route, such as injected into a vein with an IV line and slowly dripped in over time, an intramuscular route, such as injected into muscle through skin with a syringe, a topical route, such as being applied to skin, an enteric route, whereby the molecule is delivered directly into the stomach with a G-tube or J-tube, the nasal route, such as by sprays or pumps that deliver drug into the nose, the inhalation route, wherein the molecule is inhaled through a tube or mask (e.g. lung medications), the otic route, whereby drops containing the molecule may be added into the ear, an ophthalmic route, such as molecule containing drops, gel or ointment for the eye, the sublingual route, whereby the molecule is administered under the tongue, the buccal route, wherein the molecule is held inside the cheek, the transdermal route wherein the molecule is placed on a patch on the skin, or the subcutaneous route, wherein the molecule is injected just under the skin.

The attached Figures allow for a better understanding of the essence of the exemplary embodiments.

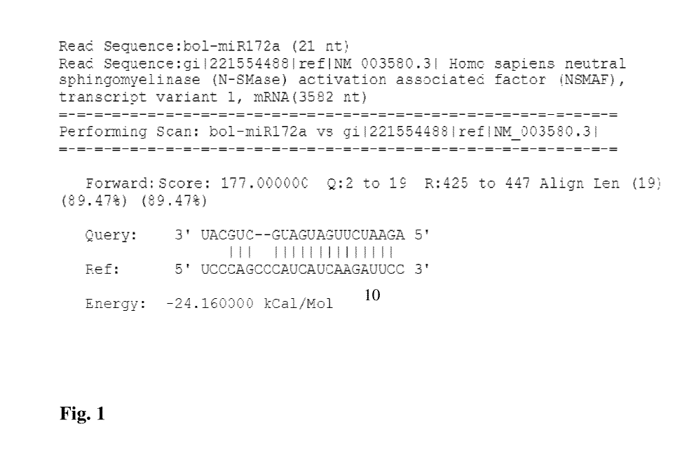

Referring now to the drawings, FIG. 1 shows the binding site of mRNA encoding FAN protein (marked as “Reference”) predicted for bol-miR172a molecule (marked as “Query”). In the alignment “|” refers to a perfect complementarity between bases, “-” represents a gap, while “:” stands for a G:U wobble pair. The calculated score for presented alignment is 177, while minimum free energy of the structure (MFE) was −24.16 kcal/mol.

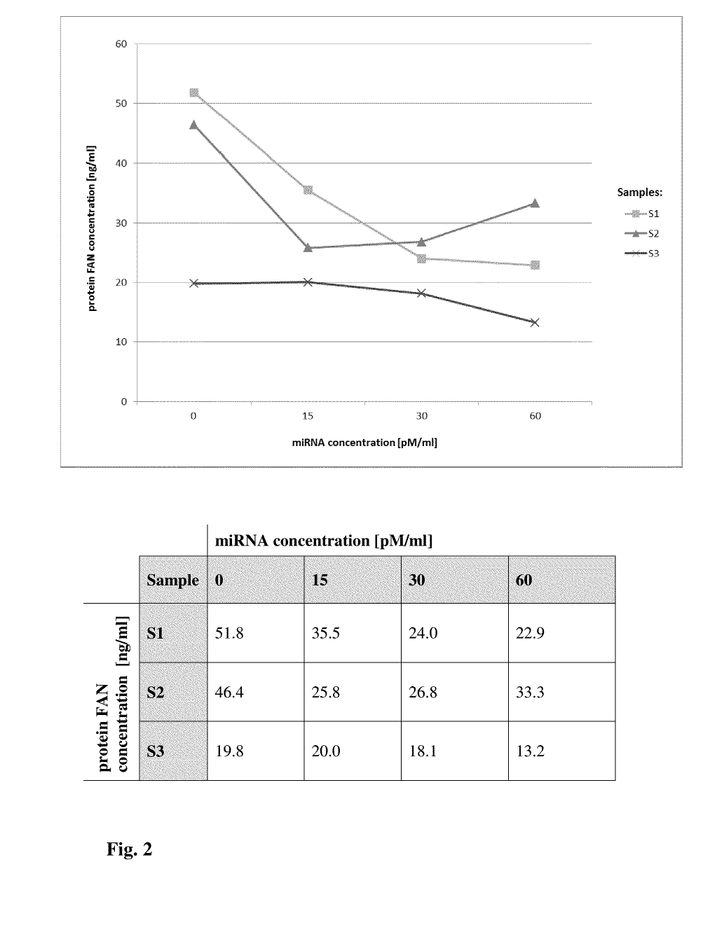

FIG. 2 shows the results of the measurements of FAN protein concentration (ng/ml) depending on the bol-miR172a molecule presence (at 3 different concentrations). The in vitro ELISA assay was performed on peripheral blood MNCs. The presented results are mean values (ng/ml) calculated from two replicates.

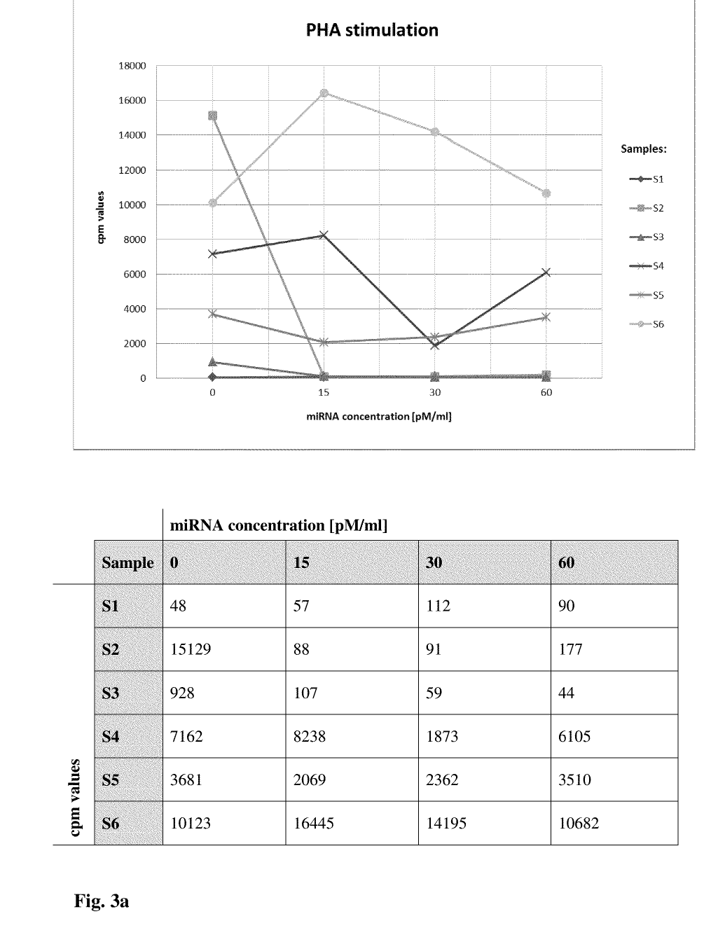

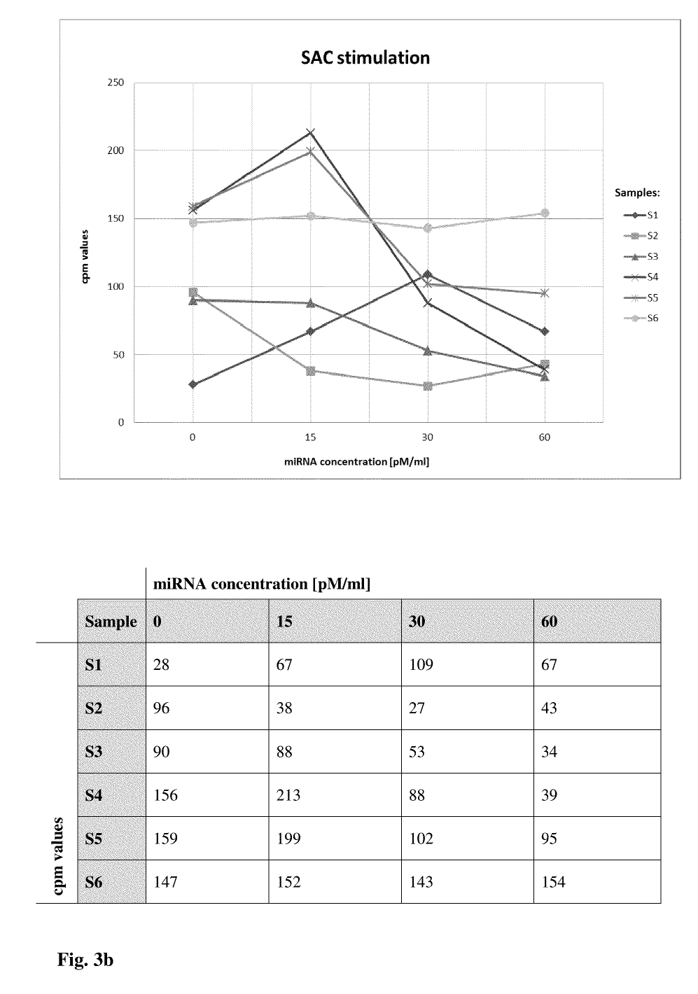

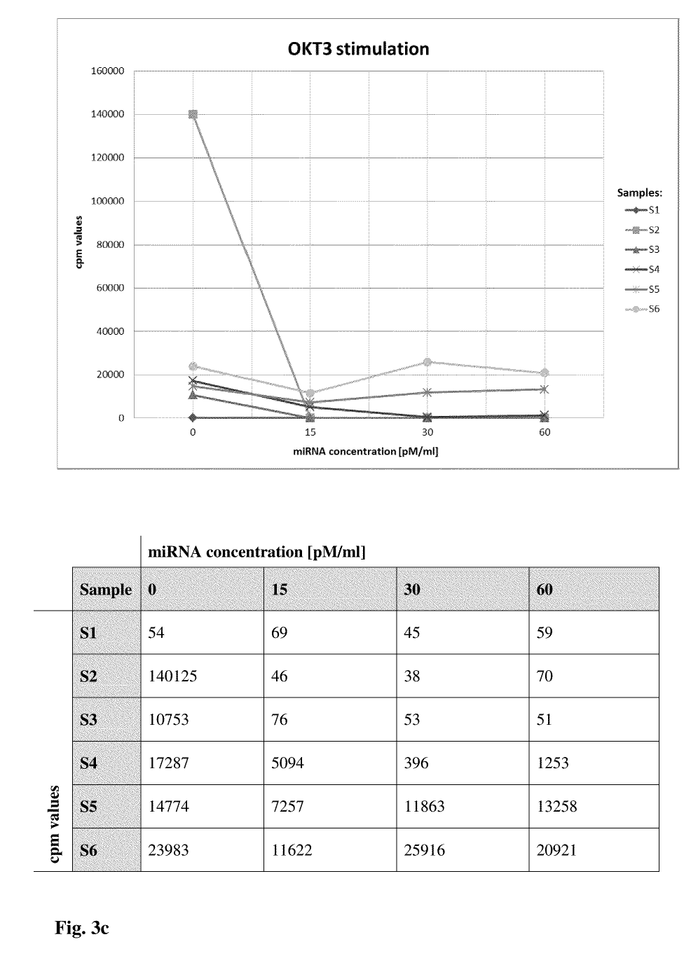

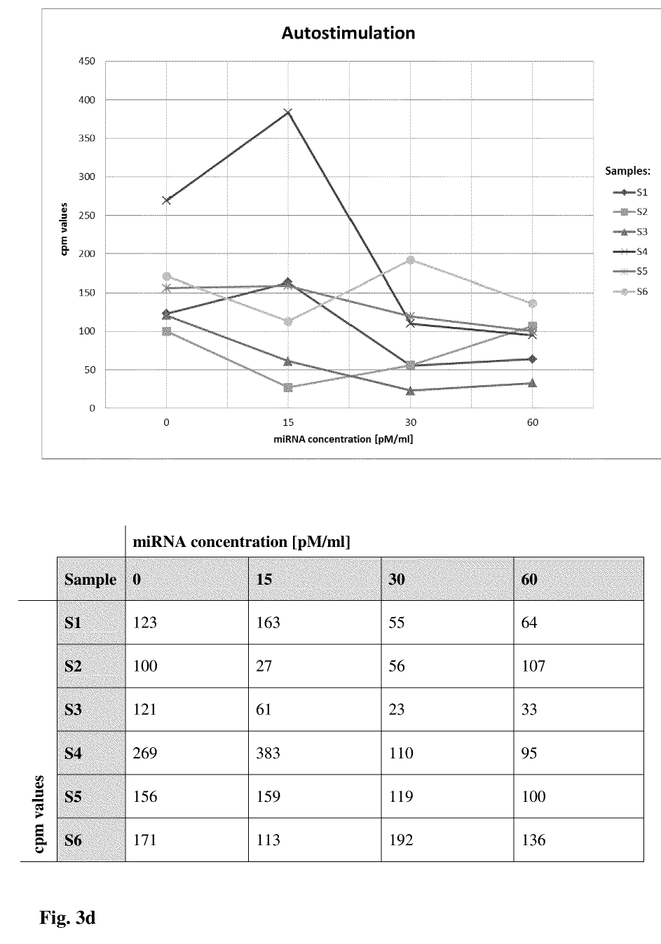

FIG. 3 shows the results of the in vitro assay for the T and B lymphocyte function, where FIG. 3a shows the influence of the bol-miR172a molecule (present at 3 different concentrations) on the proliferation of T lymphocytes stimulated by phytohemagglutinin (PHA), FIG. 3b shows the influence of the bol-miR172a (present at 3 different concentrations) on the proliferation of B lymphocytes stimulated by Staphylococcus aureus (Cowan strain, SAC), FIG. 3c shows the influence of the bol-miR172a (present at 3 different concentrations) on the proliferation of T lymphocytes stimulated by anti-CD3 antibodies (OKT3), and FIG. 3d shows the influence of the bol-miR172a (present at 3 different concentrations) on the proliferation of B lymphocytes in the case of their autostimulation. The presented results are mean cpm values calculated from three replicates.

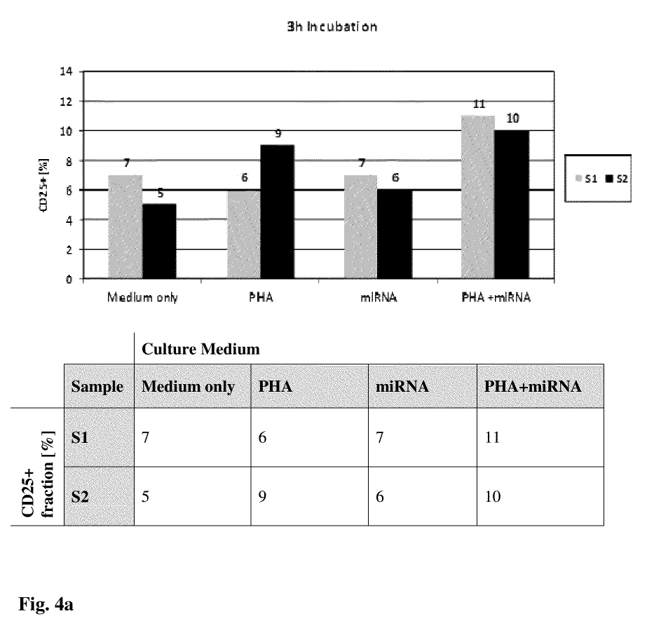

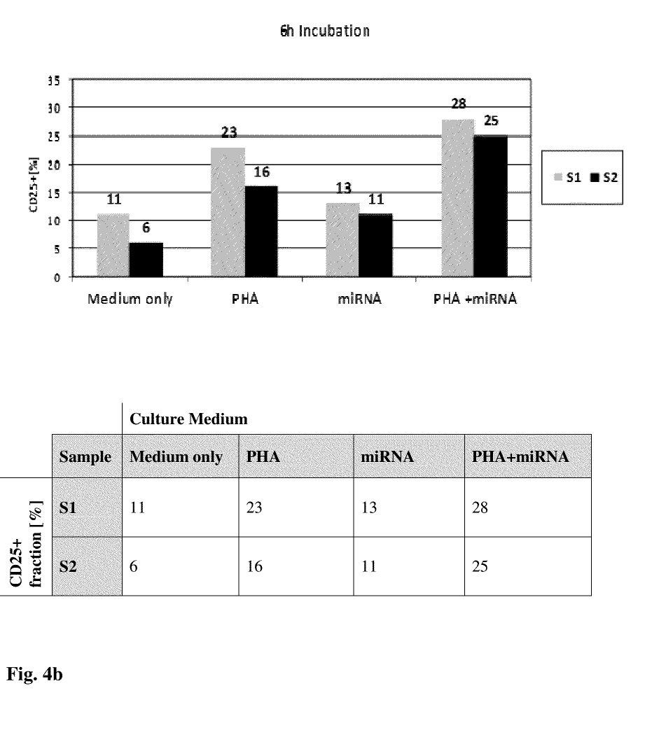

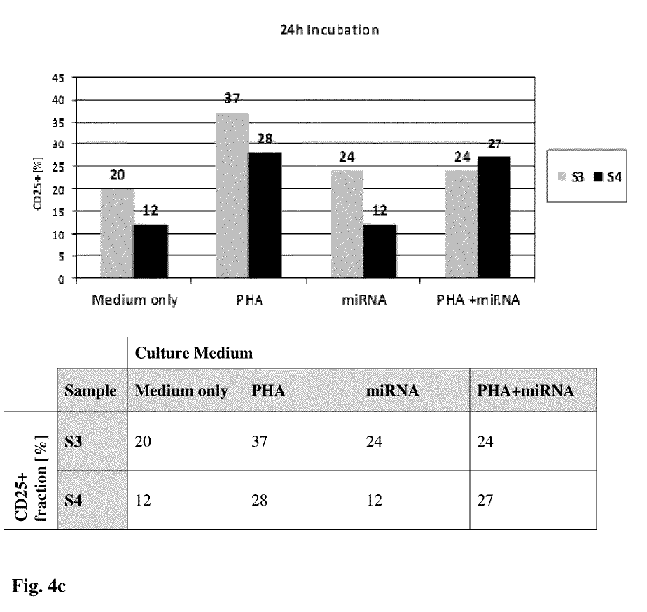

FIG. 4 shows the results of the phenotypic evaluation of PBMCs using flow cytometry, where FIG. 4a shows the results of phenotypic evaluation of MNCs incubated with miRNA and miRNA+PHA mitogen for 3 hours, FIG. 4b shows the results of phenotypic evaluation of MNCs incubated with miRNA and miRNA+PHA mitogen for 6 hours, and FIG. 4c shows the results of phenotypic evaluation of MNCs incubated with miRNA and miRNA+PHA mitogen for 24 hours. The presented results constituting the fraction (%) of CD25+ cells in lymphocyte CD3+ population are mean values calculated from three replicates.

### EXAMPLES

Exemplary embodiments of an anti-inflammatory described herein are further illustrated by the following examples, which are set forth to illustrate the presently disclosed subject matter and are not to be construed as limiting.

### Example 1

### High-Throughput Sequencing Identification of miRNA Molecules in the Mature Brassica oleracea Leaves

Using the modified Trizol method as described by Szarzynska et al., total RNA enriched with sRNA was isolated from the mature cabbage leaves (Brassica oleracea var. capitata, cultivar Balbro) in three independent biological replicates. Next generation sequencing (NGS) using the Illumina HiSeq technology was performed according to the manufacturer's protocol. Sequencing reads were generated from three constructed small RNA libraries. Each raw sRNAs dataset was further bioinformatically analyzed to clean, remove unnecessary tags and identify sequences representing conserved and novel miRNAs. The relative quantities of discovered cabbage miRNA molecules were estimated on the basis of mean values, calculated from the normalized number of miRNA tags in all three libraries. The data obtained from small RNAs sequencing have been deposited in the NCBI's Gene Expression Omnibus repository under accession number GSE45578.

### Example 2

### Prediction of Putative Human Target Genes for Brassica oleracea miRNAs

The sequences of the identified conserved and novel cabbage miRNAs were used to predict their potential human target genes. The Homo sapiens 3′UTR, 5′UTR and CDS sequences, that were downloaded from the UCSC Genome Bioinformatics Site (http://genome.ucsc.edu/) and NCBI CCDS Database (www.ncbi.nlm.nih.gov/CCDS/CcdsBrowse.cgi), respectively, served as reference target genes dataset. The bioinformatics targets prediction was performed using miRanda method (www.microrna.org/microrna/getDownloads.do). The miRanda search procedure examines sequence complementarity, interspecies conservation and thermodynamic stability of the miRNA:mRNA duplex.

The prediction parameters were as follows: (1) G:U base pairing was permitted but scored lower (score+2) than canonical base pairs (score+5), (2) the alignments with gaps and non-canonical base pairs in the “seed” regions (2-8 nt at the 5′ end of the molecule) were discarded, and (3) alignments with scores over 130 and minimum free energy (MFE) of the structure less than −17 kcal/mol were selected. The generated list of putative human targets genes was sorted by the highest alignment score and lowest MFE of the structure. To designate potential processes involving the predicted mRNA sequences and to suggest a probable influence of cabbage miRNAs on human organism, the selected targets were mapped on the UniProt database (www.uniprot.org) and annotated using the Blast2GO (http://www.blast2go.com/b2ghome) software. The obtained dataset was analyzed manually and the human target gene for cabbage bol-miR172 was selected—mRNA encoding FAN protein with the sequence SEQ. ID No. 3.

### Example 3

### Measurement of FAN Protein Quantity Using ELISA Assay

Mononuclear cells (MNCs) were isolated from peripheral blood of healthy donors by density gradient centrifugation for 12 min (2500 rpm, room temperature) using Ficoll-Histopaque (1077 g/l). Isolated MNCs were washed twice with 0.9% NaCl solution (1800 rpm, 10 min) and cultured in Parker medium supplemented with L-glutamine (2 mM), 3-β-mercaptoethanol, HEPES (0.23%), fetal calf serum (FCS, 10%) and gentamicin (0.1 mg/ml). The appropriate volume of miRNA was added to the cultures to yield a 15 pM, 30 pM and 60 pM concentrations. LPS in a 1 μg/ml concentration (Sigma-Aldrich) was used to stimulate cells. After addition of LPS, cells were incubated at 37° C., in 5% CO₂ humidified atmosphere for 72 h. Next, cells were collected, centrifuged (1800 rpm, 10 min) and washed twice with phosphate-buffered saline (PBS). Centrifuged cells were resuspended in 200 μl of PBS and frozen in −20° C. According to the ELISA kit manufacturer's protocol (Cloud-Clone Corp.), cells were frozen and thawed three times before FAN protein labelling. The level of FAN protein was determined in the collected supernatant using the LEDETECT96 plate reader at 450 nm wavelength.

### Example 4

### In Vitro Proliferation Assay for Lymphocyte Function

### Peripheral Blood Mononuclear Cells (PBMCs) Isolation:

Mononuclear cells (MNCs) were isolated from peripheral blood of healthy donors by gradient centrifugation using Gradisol (Ficoll) (1077 g/l). Next, they were counted in a Bürker chamber using Turk solution. Isolated MNCs were diluted to 1×10⁶ cells/ml concentration in culture medium. The cell lines were cultured in Parker medium supplemented with L-glutamine (2 mM), 3-β-mercaptoethanol, HEPES (0.23%), fetal calf serum (FCS, 10%) and gentamicin (0.1 mg/ml).

### Proliferation Test:

The lymphocytes cultures were seeded into a 96-well plate with 1×10⁵ cells/well and stimulated by specific mitogens: anti-CD3 monoclonal antibodies (OKT3, 1 μg/ml), phytohemagglutinin (PHA, 20 μg/ml, Sigma-Aldrich) and Staphylococcus aureus Cowan strain (SAC, 0.004%) suspension. To each well the appropriate volume of miRNA was added to yield a 15 pM, 30 pM and 60 pM concentrations. The control cultures contained the equivalent volume of culture medium only. The prepared cultures were incubated at 37° C., in 5% CO₂ humidified atmosphere for 72 h. After addition of the [³H] thymidine, incubation was continued for 17 h in the aforementioned conditions. The cell cultures with [³H] thymidine incorporated into DNA, were transferred from plate onto filter paper by automatic harvester. For cell counting the scintillation counter was used. The results are reported as counts per minute (cpm).

### Example 5

### Evaluation of PBMCs Phenotype Under PHA Mitogen Stimulation and miRNA Abundance

The evaluation of CD25+ (a chain of IL-2 receptor) cells subpopulation in MNCs population was performed with the use of immunofluorescence technique and flow cytometry. Mononuclear cells (MNCs) were isolated from peripheral blood of healthy donors by gradient centrifugation using Gradisol L (1077 g/l). Next, the isolated MNCs (in 1×10⁶ cells/ml concentration) were incubated for 3 h, 6 h and 24 h with 30 pM/ml miRNA, after PHA (Sigma-Aldrich) stimulation in 20 μg/ml and 30 μg/ml concentration. Cell's phenotype was labelled with the appropriate monoclonal antibodies CD25-FITC and CD3-PE (Becton-Dickinson) by bicolor immunofluorescence technique. The MNCs were incubated with the antibodies for 15 min in room temperature, without access to the light. The labeled cells were then washed twice with PBS supplemented with 0.1% FCS. Next, they were resuspended in 500 μl of FACSFlow and detected by the flow cytometer FACSCalibur (Becton-Dickinson). Data acquisition, storing and analysis was performed with the use of CellQuest software (Becton-Dickinson). The results were presented as CD25+ cells fraction in CD3+ cells (lymphocytes T) population.

### Example 6

### Identification of miRNA Molecules in Mature Cabbage Leaves

To identify a larger set of miRNA molecules in the mature Brassica oleracea var. capitata leaves the high-throughput sequencing of three independent sRNA libraries was performed. The generated raw reads were further bioinformatically processed. As a result of aforementioned analysis, 261 conserved miRNAs (belonging to 62 families) were discovered (together with their estimated quantities) [46]. Among the most abundant molecules, the bol-miR172a (SEQ. ID No. 1) and bol-miR172b (SEQ. ID No. 2), were found.

### Example 7

### Potential Human Target Genes Prediction for Identified Brassica oleracea miRNAs

Predictions of putative human target genes for cabbage miRNAs molecules were performed. The results generated by miRanda method were sorted by the highest alignment score and lowest MFE of the structure. Among the predicted human targets the most associated with the inflammatory processes, was the mRNA molecule encoding FAN protein (SEQ. ID No. 3). Molecules bol-miR172a (SEQ. ID No. 1) and bol-miR172b (SEQ. ID No. 2), which are present in a large quantities in cabbage leaves, were selected to interact with the mRNA encoding FAN protein. The aforementioned hybridization occurs, according to the method used, in the CDS region of the target mRNA. The sequence complementarity of the miRNA and mRNA binding site was designated to 89.4%. The calculated alignment score was 177, while minimum free energy of the structure was −24.16 kcal/mol (FIG. 1).

### Example 8

### Influence of miRNA Abundance on FAN Protein Level

An important step in implementation was to determine the impact of the molecule bol-miR172a or bol-miR172b on the FAN protein level. Performed on the PBMCs, in vitro analysis with the use of ELISA method showed that abundance of bol-miR172a in a concentration of 15 pM, 30 pM, as well as 60 pM reduces significantly level of the FAN protein in analyzed preparation. The greatest decrease of FAN protein quantity was observed in a presence of bol-miR172a in 30 pM concentration (FIG. 2).

### Example 9

### Effect of miRNA Abundance and Mitogen Stimulation on T and B Lymphocyte Proliferation

In vitro evaluation of the T and B lymphocyte function associated with their autostimulation or stimulation by specific mitogen, such as PHA, SAC and OKT3, showed that abundance of bol-miR172a in 30 pM/ml concentration significantly reduce the B and T lymphocyte proliferation in the case of their autostimulation or presence of a specific mitogen (FIGS. 3a, b, c and d).

### Example 10

### Phenotypic Evaluation of MNCs by Flow Cytometry

Flow cytometric analysis of PBMCs incubated for 3 h, 6 h and 24 h with bol-miR172a molecule (in 30 pM/ml concentration), confirmed the slightly altered fraction of CD25+ cells in lymphocyte CD3+ population relative to the control. Additionally, similar minor changes in fraction of CD25+ cells in lymphocyte CD3+ population relative to the control were observed in the case of the simultaneous presence of bol-miR172a molecule (30 pM/ml) and PHA mitogen in 30 μg/ml concentration (FIGS. 4a, b and c).

Of course these described embodiments are exemplary and alterations thereto are possible by those having skill in the relevant technology.

Thus the example embodiments and arrangements achieve improved capabilities, eliminate difficulties encountered in the use of prior compositions and methods, and attain the desirable results described herein.

In the foregoing description, certain terms have been used for brevity, clarity and understanding. However, no unnecessary limitations are to be implied therefrom because such terms are used for descriptive purposes and are intended to be broadly construed.

Moreover the descriptions and illustrations herein are by way of examples and the embodiments not limited to the features shown and described.

Further, it should be understood that components, materials, features and/or relationships associated with one embodiment can be combined with components, materials, features and/or relationships from other embodiments. That is, various components, materials, features and/or relationships from various embodiments can be combined in further embodiments. The inventive scope of the disclosure is not limited to only the embodiments shown or described herein.

Having described the features, discoveries and principles of the exemplary embodiments, the manner in which they are made, utilized and carried out, and the advantages and useful results attained, the new and useful articles, arrangements, combinations, methodologies, structures, devices, elements, combinations, operations, processes and relationships are set forth in the appended claims.

SEQUENCE LISTING

miR172a, RNA molecule, 21 nt
SEQ. ID No. 1
AGAAUCUUGAUGAUGCUGCAU
miR172b, RNA molecule, 21 nt
SEQ. ID No. 2
AGAAUCUUGAUGAUGCUGCAU
NM_003580.3, Homo sapiens neutral sphingomyelinase
(N-SMase) activation associated factor (NSMAF),
transcript variant 1, mRNA, 3582 bp
SEQ. ID No. 3
GCCGGAGTCCCCACGCGAGGATGCTGCGGTGAGCGCGGCCGGGACGCCGC
GTCGGGCTCTCGGCCGCCCAGCGGCGCCGCAGGGGAAGCCAGGCCGGCAG
CCGCGCGCTCCCCAGCCGGCCACCAATCCCGCCCTCCCCGGCTCCTCCGG
ACCTCCTCGGCAGGCGGCGGCGGGGCCTGCCTGTGCGCTCTGCGCCCGCC
CGCGCCTACCCTCCATGGCGTTTATCCGGAAGAAGCAGCAGGAGCAGCAG
CTGCAGCTCTACTCCAAGGAGAGATTTTCCTTGCTGCTGCTTAACTTGGA
GGAGTACTACTTTGAACAGCATAGAGCCAATCACATTTTGCACAAGGGCA
GTCACCATGAAAGGAAAATCAGAGGCTCCTTAAAAATATGTTCAAAATCG
GTGATTTTTGAACCAGATTCAATATCCCAGCCCATCATCAAGATTCCTTT
GAGAGACTGTATAAAAATAGGAAAGCATGGAGAAAATGGAGCCAATAGAC
ACTTCACAAAGGCAAAATCTGGGGGTATTTCACTCATTTTCAGTCAGGTA
TATTTCATTAAAGAACATAATGTTGTTGCACCATATAAAATAGAAAGGGG
CAAAATGGAATATGTTTTTGAATTGGATGTTCCCGGGAAAGTGGAAGATG
TTGTGGAGACGTTGCTTCAGCTTCACAGAGCATCCTGCCTTGACAAATTG
GGTGACCAAACCGCCATGATAACAGCTATTTTGCAGTCTCGTTTAGCTAG
AACATCATTTGACAAAAACAGGTTCCAAAACATTTCTGAAAAGCTGCACA
TGGAATGCAAAGCAGAAATGGTGACGCCTCTGGTGACTAATCCTGGACAC
GTGTGCATCACGGACACAAACCTGTATTTTCAGCCCCTCAACGGCTACCC
GAAACCTGTGGTCCAGATAACACTCCAAGATGTCCGCCGCATCTACAAAA
GGAGGCACGGCCTCATGCCTCTGGGCTTGGAAGTATTTTGCACAGAAGAT
GATCTGTGTTCCGACATCTACCTAAAGTTCTATGAACCTCAAGATAGAGA
TGATCTCTATTTTTACATTGCCACATACCTAGAGCACCATGTGGCGGAGC
ACACTGCTGAGAGCTACATGCTGCAGTGGCAGCGTGGACACCTTTCCAAC
TATCAGTACCTCCTTCACCTCAACAACCTGGCCGACCGCAGCTGCAACGA
CCTCTCCCAGTACCCTGTGTTTCCATGGATAATACATGATTATTCCAGCT
CAGAACTAGATTTGTCAAATCCAGGAACCTTCCGGGATCTCAGTAAGCCA
GTAGGGGCCCTAAATAAGGAACGGCTGGAGAGACTACTGACACGCTACCA
GGAAATGCCTGAACCAAAGTTCATGTATGGGAGTCACTACTCTTCCCCGG
GTTATGTACTTTTTTATCTTGTTAGGATTGCACCAGAGTATATGCTGTGC
CTGCAGAATGGAAGATTTGATAATGCAGATAGAATGTTCAACAGTATTGC
AGAAACTTGGAAAAACTGTCTGGATGGTGCAACGGATTTTAAAGAGTTAA
TTCCAGAATTCTATGGTGATGATGTGAGCTTTCTAGTCAATAGCCTGAAG
TTGGATTTGGGAAAGAGACAAGGAGGACAGATGGTTGACGACGTGGAGCT
TCCCCCTTGGGCTTCCAGTCCCGAGGACTTTCTCCAGAAGAGCAAAGATG
CATTGGAAAGCAATTATGTGTCTGAACACCTTCACGAGTGGATTGATCTA
ATATTTGGCTACAAACAAAAAGGGAGTGATGCAGTTGGGGCCCATAATGT
ATTTCATCCCCTGACCTATGAAGGAGGTGTAGACTTGAACAGCATCCAGG
ATCCTGATGAGAAGGTAGCCATGCTTACGCAAATCTTGGAATTTGGGCAG
ACACCAAAACAACTATTTGTGACACCACATCCTCGAAGGATCACCCCAAA
GTTTAAAAGTTTGTCCCAGACCTCCAGTTATAATGCTTCTATGGCAGATT
CCCCAGGTGAAGAGTCTTTTGAAGACCTGACCGAAGAAAGCAAAACACTG
GCCTGGAATAACATCACCAAACTGCAGTTACACGAGCACTATAAAATCCA
CAAAGAAGCAGTTACTGGAATCACGGTCTCTCGCAATGGATCTTCAGTAT
TCACAACATCCCAAGATTCCACCTTGAAGATGTTTTCTAAAGAATCAAAA
ATGCTACAAAGAAGTATATCATTTTCAAATATGGCTTTATCGTCTTGTTT
ACTTTTACCAGGAGATGCCACTGTCATAACTTCTTCATGGGATAATAATG
TCTATTTTTATTCCATAGCATTTGGAAGACGCCAGGACACGTTAATGGGA
CATGATGATGCTGTTAGTAAGATCTGTTGGCATGACAACAGGCTATATTC
TGCATCGTGGGACTCTACAGTGAAGGTGTGGTCTGGTGTTCCTGCAGAGA
TGCCAGGCACCAAAAGACACCACTTTGACTTGCTGGCCGAGCTGGAACAT
GATGTCAGTGTAGATACAATCAGTTTAAATGCTGCAAGCACACTGTTAGT
TTCCGGCACCAAAGAAGGCACAGTGAATATTTGGGACCTCACAACGGCCA
CCTTAATGCACCAGATTCCATGCCATTCAGGGATTGTATGTGACACTGCT
TTTAGCCCAGATAGTCGCCATGTCCTCAGCACAGGAACAGATGGCTGTCT
TAATGTCATTGATGTGCAGACAGGAATGCTCATCTCCTCCATGACATCAG
ATGAGCCCCAGAGGTGCTTTGTCTGGGATGGAAATTCCGTTTTATCTGGC
AGTCAGTCTGGTGAACTGCTCGTTTGGGACCTCCTTGGAGCAAAAATCAG
TGAGAGAATACAGGGCCACACAGGTGCTGTGACATGTATATGGATGAATG
AACAGTGTAGCAGTATCATCACAGGAGGGGAAGACAGACAAATTATATTC
TGGAAATTGCAGTATTAAGTGCCTTTTCCTCTCCTGAATATTAAATTGAA
CTCTATTTAATGCATTTTTAAACCAAACTTTTAAACGGACTGGTGAATGT
GCAATGTTAGTAATTAGAAGTTTTACCACATGGAAAATTTGTGGTTTTAA
ACTTTCTAAATCATGGTGACTTCATTGAAAGCCATTAGTTGCTATTCTCT
TAGGGCAGATAAAATGCGGCTGTGTTAGGAAAAACATGTTACACTGTAAG
GCAGATGATCGTCCCCGTATGATGATTGTCAGAAGACAGGACTAAGTAGC
AGAGAATAGCTAAGAGATAAATTGGGCTGGGGAAACTTGTCAGAAAGCAC
TGAACAATTAAGAAATTTTCCAAGAAAATGTGCAGTATTCTCTGCTACTT
CTGAATCTGTTTTGTCTTCCTAATCTATCACAATTGCCACCCATCGGGTT
TTGGGTGTGTGTTTTCATAGCGTGGTTACTTTCTATAATGCTGTACCCAG
ATTCTAAGAACCTGGAGAAGGATTAGCAGTTCTTAGTAAGTTTACTGTGT
ATAGGAACGGTTTGTATTTCATTACAGCTATTCATCTTTTCTACATTAAA
AATATTTTTCTCTAAAGAAAAAAAAAAAAAAA

We claim:

1. A method comprising:
administering to a living organism a plant-derived miR172 molecule or its synthetic equivalent,
wherein the molecule is selected from amongst miR172a or miR172b,
wherein miR172a is represented by the sequence SEQ. ID No. 1, and
wherein miR172b by the sequence SEQ. ID No. 2,
wherein the molecule has at least 6 out of 7 nucleotides present in a seed region represented by the sequence GAAUCUU,
wherein the molecule has at least 75% sequence similarity to SEQ. ID No. 1 or SEQ. ID No. 2,
in an amount sufficient to at least one of:
decrease or
limit increase,
of an inflammatory response of the living organism.

2. The method of claim 1, wherein the plant-derived miR172 molecule or its synthetic equivalent reduces proliferation of T and B lymphocytes.

3. The method of claim 1, wherein the plant-derived miR172 molecule or its synthetic equivalent reduces the level of FAN (Factor Associated with Neutral Sphingomyelinase Activation) protein.

4. The method of claim 1, wherein the plant-derived miR172 molecule or its synthetic equivalent interacts with a mRNA encoding a FAN protein,
whereby the plant-derived miR172 molecule or its synthetic equivalent negatively regulates the mRNA expression and reduces proliferation of T and B lymphocytes.

5. The method of claim 1, wherein a plant-derived miR172 molecule or its synthetic equivalent interacts with a mRNA encoding a FAN protein by negatively regulating its expression,
whereby the plant-derived miR172 molecule or its synthetic equivalent reduces the level of the FAN protein.

6. The method of claim 5, wherein the expression of FAN protein is negatively regulated and the level of FAN protein is reduced.

7. The method of claim 1, wherein the plant-derived miR172 molecule or its synthetic equivalent is 6, 7, 8, 9, 10, 11, 12, 13, 14, 15, 16, 17, 18, 19, 20, 21, 22, 23, 24, 25, 26, 27 or 28 nucleotides long.

8. The method of claim 1, wherein plant-derived miR172 molecule or its synthetic equivalent has 2′- or 3′-O-methylation of the ribose of last nucleotide at the 3′ end.

9. The method of claim 1, wherein the inflammatory response of the organism is decreased by reducing proliferation of T and B lymphocytes.

10. The method of claim 1, wherein the inflammatory response of the organism is decreased by reducing the level of FAN protein.

11. A method of treating autoimmune diseases or disorders in a subject in need thereof, wherein the method comprises:
administering to the subject a therapeutically effective amount of a plant based miR-172a or miR-172b or their synthetic equivalents comprising the sequence as identified by SEQ ID NO:1 in the case of miR-172a, or its synthetic equivalent, or SEQ ID NO:2 in the case of miR-172b or its synthetic equivalent.

12. The method of claim 11, wherein plant based miR-172a or miR-172b or their synthetic equivalents molecule reduces proliferation of T and B lymphocytes.

13. The method of claim 11, wherein plant based miR-172a or miR-172b or their synthetic equivalents reduces the level of FAN (Factor Associated with Neutral Sphingomyelinase Activation) protein.

14. The method of claim 11, wherein plant based miR-172a or miR-172b or their synthetic equivalents interacts with a mRNA encoding a FAN protein,
plant based miR-172a or miR-172b or their synthetic equivalents, negatively regulate the mRNA expression and reduce proliferation of T and B lymphocytes.

15. The method of claim 11, wherein plant based miR-172a or miR-172b or their synthetic equivalents interacts with a mRNA encoding a FAN protein by negatively regulating its expression,
whereby, plant based miR-172a or miR-172b or their synthetic equivalents reduces the level of the FAN protein.

16. The method of claim 15, wherein the expression of FAN protein is negatively regulated and the level of FAN protein is reduced.

17. The method of claim 11, wherein plant based miR-172a or miR-172b or their synthetic equivalents are 6, 7, 8, 9, 10, 11, 12, 13, 14, 15, 16, 17, 18, 19, 20, 21, 22, 23, 24, 25, 26, 27 or 28 nucleotides long.

18. The method of claim 11, wherein plant based miR-172a or miR-172b or their synthetic equivalents has 2′- or 3′-O-methylation of the ribose of last nucleotide at the 3′ end.

19. The method of claim 11, wherein the inflammatory response of the organism is decreased by reducing proliferation of T and B lymphocytes.

20. The method of claim 11, wherein the inflammatory response of the organism is decreased by reducing the level of FAN protein.
